(19) United States
(12) Patent Application Publication
(10) Pub. No.: US 2004/0049823 A1
(43) Pub. Date: Mar. 11, 2004

(54) Altered flower pattern and color in new guinea impatiens

(75) Inventors: Scott Trees, Nipomo, CA (US); Andreas Olbring, Nipomo, CA (US)

Correspondence Address: WOOD, PHILLIPS, KATZ, CLARK & MORTIMER, 500 W. MADISON STREET, SUITE 3800, CHICAGO, IL, 60661, US

(73) Assignee: Ball Horticultural Company.

(21) Appl. No.: 10/660,999

(22) Filed: Sep. 12, 2003

Related U.S. Application Data
(63) Continuation of application No. 09/488,296, filed on Jan. 20, 2000.
(60) Provisional application No. 60/116,549, filed on Jan. 21, 1999.

(51) Int. Cl. A01H 5/00

(52) U.S. Cl. 800/323

(57) ABSTRACT

The present invention relates to NGI plants which produce an inflorescence containing one or more flowers which have at least one petal per flower which exhibits a striped pattern or altered flower color and methods for making such plants.

### RELATED APPLICATION INFORMATION

This application is a continuation-in-part of U.S. Application No. 60/116,549 filed on Jan. 21, 1999.

### FIELD OF THE INVENTION

The present invention relates to new, distinct and stable New Guinea impatiens plants (hereinafter referred to as “NGI”) exhibiting a striped pattern or an altered flower color. The NGI plants of the present invention produce an inflorescence containing one or more flowers which have at least one petal per flower which exhibits a striped pattern or an altered flower color. The present invention also relates to methods for making a New Guinea impatiens plants having an inflorescence containing one or more flowers which have at least one petal per flower which exhibits a striped pattern or an altered flower color.

### BACKGROUND OF THE INVENTION

Only a very few floricultural crops have been introduced to the floriculture industry in the last 25 years that can compare with the success of New Guinea impatiens. (New Guinea Impatiens, A Ball Guide, edited by W. Banner and M. Klopmeyer, Ball Publishing (1995)). Initially, the NGI was regarded primarily as a bedding plant. Id. Now the crop is no longer confined to a bedding plant classification. Id. Instead, growers have realized that NGIs can be used as flowering potted plants and in hanging baskets. Id.

Impatiens plants reported to be native to New Guinea were classified in 1886 as Impatiens Hawkeri. Id. Occasionally, these early specimens were referred to as Impatiens herzogii. Id. In the early 1900's, botanists from Germany, England and the Netherlands explored parts of the Sundra Islands, and by 1915 nine New Guinea impatiens species were identified from this area: I dahili, I herzogii, I laxterbachii, I linearifolia, I inooreana, I. polyphylla, I rodatzii, I schlechteri, and I. trichura. Id. Taxonomically the collections were confusing and were considered to be habitat variations of I. herzogii rather than new species by Von R. Schlecter. Id. Expeditions by botanists from the United States, Australia, France and Switzerland also searched Papua and the New Guineas for new impatiens species. Id. In the most recent taxonomic classification, Grey-Wilson proposed that New Guinea impatiens belong to one highly variable species, I. Hawkeri, in which 15 groups were identified based on geographic location. Id.

Although diverse phenotypically, members of New Guinea impatiens are interfertile and generally have a 2n chromosome number of 32 (T. Arisumi, J Hered., 64: 77-79 (1973)). In 1970, the U.S. Department of Agriculture introduced 23 impatiens from New Guinea, in order to increase the germplasm pool for this crop. (H.F. Winters, Am. Hortic., 52, 923 (1973)). Breeding programs initiated with this material have led to the development of New Guinea impatiens plants that are adapted to a variety of light conditions, and have large flowers of a wide variety of colors including white, pink, red, orange, purple, rose, salmon and coral as well as bicolors. (New Guinea Impatiens, A Ball Guide, edited by W. Banner and M. Klopmeyer, Ball Publishing (1995)).

### SUMMARY OF THE INVENTION

The present invention relates to a NGI plant which has a flower containing at least one petal which exhibits a striped pattern or an altered flower color. This NGI plant may have a pedigree which includes the plants 1865, 2581 or 2582. Additionally, the present invention also includes pollen, ovules and seed from such a striped or altered flower color NGI plant. Moreover, the present invention also relates to a tissue culture of regenerable cells as well as a cutting from such a striped or altered flower color NGI plant.

The present invention also relates to a method for producing a NGI plant having a flower with at least one petal which exhibits a striped pattern. The method involves irradiating a NGI plant with electromagnetic radiation, ion beams or treating a NGI plant with a chemical mutagen and then selecting a shoot from the treated plant containing a flower having at least one petal which exhibits a striped pattern. The irradiated or chemically treated plant may be a rooted cutting or a whole plant.

If irradiated, the plant may be irradiated with gamma rays, x-rays or ultraviolet rays. The NGI plant may be irradiated with from about 1.5 to about 3.5 krads of electromagnetic radiation or ion beams.

If the NGI plant is treated with a chemical mutagen, it may be treated with alkylating sulphonates and sulphates, such as ethyl methane sulphonate, methyl methane sulphonate and diethyl sulphate, or nitroso compounds, such as nitrosoguanidine, ethylnitrosourea and methylnitrosourea.

Finally, the present invention relates to a method for altering the color of the petals of a flower of a NGI plant. The method involves irradiating a NGI plant with electromagnetic radiation, ion beams or treating a NGI plant with a chemical mutagen and then selecting a shoot from the treated plant containing a flower having at least one petal which exhibits an altered flower color. The irradiated or chemically treated plant may be a rooted cutting or a whole plant.

If irradiated, the plant may be irradiated with gamma rays, x-rays or ultraviolet rays. The NGI plant may be irradiated with from about 1.5 to about 3.5 krads of electromagnetic radiation or ion beams.

If the NGI plant is treated with a chemical mutagen, it may be treated with alkylating sulphonates and sulphates, such as ethyl methane sulphonate, methyl methane sulphonate and diethyl sulphate, or nitroso compounds, such as nitrosoguanidine, ethylnitrosourea and methylnitrosourea.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1 shows a photograph of a flower from a NGI plant named ‘Grape Crush’. ‘Grape Crush’ has large round vibrant purple flowers with a red eye. The petals are brighter than Red-Purple Group 74A with the lower four petals commonly displaying bases (“displaying bases” as used herein refers to the area of common connection between the lower four petals) of Red Group 53C (adaxial), and Red-Purple Group 74B (abaxial).

FIG. 2 shows a photograph of a flower from NGI plant 1865 of the present invention. The plants of the present invention contain petals which exhibit a striped pattern. The petals have a color of Red-Purple Group 74A (adaxial) and Red-Purple Group 74B (abaxial). The stripes have a color of Red-Purple Group 65C.

FIG. 3 shows a photograph of flowers from a NGI plant named ‘Lavender Glow’. ‘Lavender Glow’ has large round purple flowers having a petal color of Purple-Violet Group 81C (adaxial) and Purple-Violet Group 81D (abaxial).

FIG. 4 shows a photograph of flowers from NGI plant 2581 of the present invention. The flowers of the plants of the present invention contain petals which exhibit a striped pattern. The petals have a color of Purple-Violet Group 8 1C (adaxial) and Purple-Violet Group 81D (abaxial). The stripes have a color of Purple Group 75D.

FIG. 5 shows a photograph of flowers from NGI plant 2582 of the present invention. The flowers of the plant of the present invention have altered flower color and have petals of color Purple Group 75D with Red-Purple Group 64C at the base (adaxial). Purple Group 75D with Red-Purple Group 64C at the base with midveins of Red-Purple Group 64B (abaxial).

### DETAILED DESCRIPTION OF THE INVENTION

The color chart used in the identification of colors described herein is the R.H.S. Colour Chart of The Royal Horticultural Society, London, England.

The NGI plants of the present invention have an inflorescence containing one or more flowers which have at least one petal per flower which exhibits a striped pattern or altered flower color. As used herein, the term “striped pattern” or “striping pattern” means that a darker background color on a flower petal is randomly interdispersed with irregular bands or streaks of a lighter color. As used herein, the term “altered flower color” means that the color on a flower petal has been changed (either lightened or darkened), when compared to a control plant not treated pursuant to the methods hereinafter described.

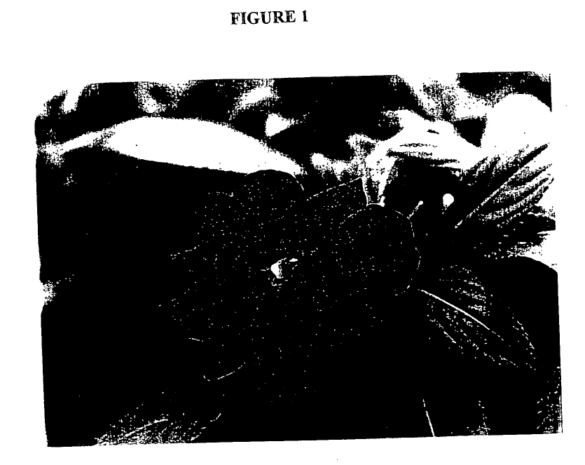

FIG. 1 is a photograph of a flower from a NGI plant named ‘Grape Crush’ (U.S. Plant Pat. No. 10,107). ‘Grape Crush’ has large round vibrant purple flowers with a red eye which displays an iridescent appearance. The petals are brighter than Red-Purple Group 74A with the lower four petals commonly displaying bases of Red Group 53C (adaxial), and Red-Purple Group 74B (abaxial). In addition, the foliage is medium green in coloration. Additionally, ‘Grape Crush’ exhibits good basal branching and an attractive moderately compact upright mounded growth habit.

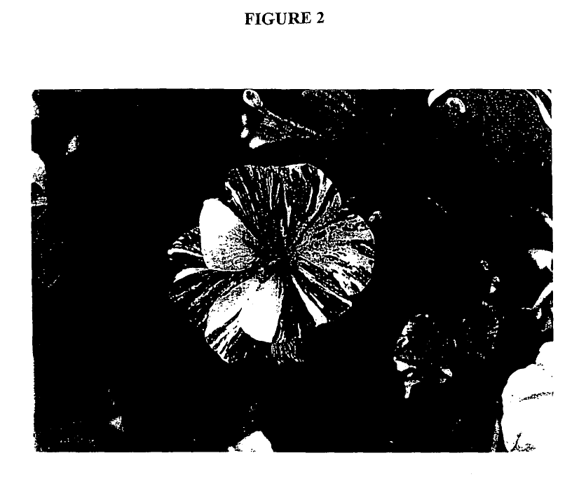

In contrast, FIG. 2 is a photograph of a flower from the NGI 1865 of the present invention. FIG. 2 shows the distinct striping of the petals of the NGI plants of the present invention. The petals have a color of Red-Purple Group 74A (adaxial) and Red-Purple Group 74B (abaxial). The stripes have a color of Red-Purple Group 65C.

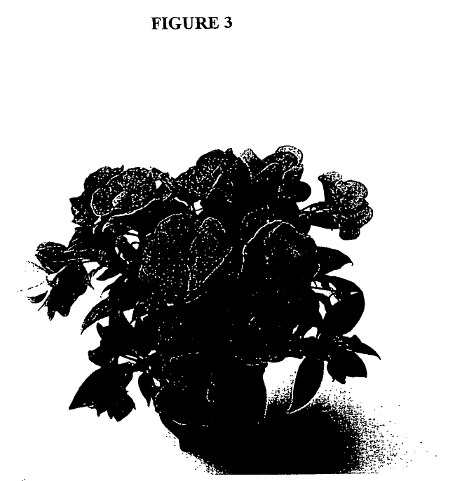

FIG. 3 shows a photograph of flowers from a NGI plant named ‘Lavender Glow’. Lavender Glow has large round purple flowers having a petal color of Purple-Violet Group 81C (adaxial) and 81D (abaxial).

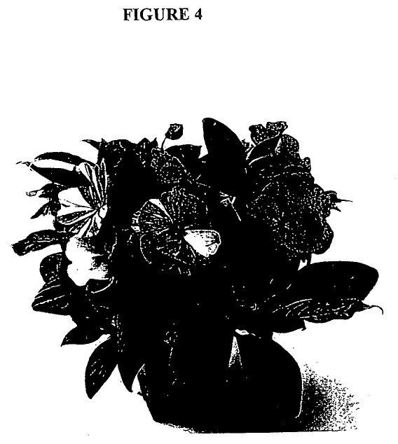

FIG. 4 shows a photograph of flowers from NGI plant 2581 of the present invention. The flowers of the plant of the present invention contain petals which exhibit a striped pattern. The petals have a color of Purple-Violet Group 81C (adaxial) and Purple-Violet Group 81D (abaxial). The stripes have a color of Purple Group 75D.

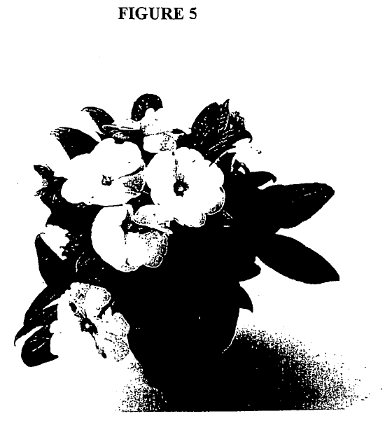

FIG. 5 shows a photograph of flowers from NGI plant 2582 of the present invention. The flowers of the plant of the present invention have petals of color Purple Group 75D with Red-Purple Group 64C at the base (adaxial). Purple Group 75D with Red-Purple Group 64C at the base with midveins of Red-Purple Group 64B (abaxial).

This previously unknown striped and altered flower color NGI plants of the present invention arose as a result of the irradiation of a NGI plant with electromagnetic radiation, particularly, gamma rays. It is believed that the irradiation of a NGI causes a somatic mutation in the cells which produce flower color and thus results in a chimera having flowers with striped petals or flowers having altered color. Additionally, the striped NGI plants of the present invention maintain functional male and female organs.

It is believed that the striped pattern and altered flower color in the petals of a NGI plant can also be obtained in a NGI plant by irradiating a plant with other types of electromagnetic radiation such as x-rays, ultraviolet rays as well as with ion beams. The NGI plant can be irradiated with from about 1.5 to about 3.5 kilorads of electromagnetic radiation or ion beams. Preferably, the NGI plants are irradiated with from about 2.0 to 3.0 kilorads of radiation or ion beams.

Additionally, the striped and altered flower color NGI plants of the present invention may also be produced by treating a NGI plants with a chemical mutagen. Like the irradiation described earlier, it is believed that treating a NGI with a chemical mutagen causes a somatic mutation in the cells which produce flower color and thus results in a chimera having flowers with striped petals or altered flower color. Any chemical mutagen which causes a somatic mutation can be used to produce the striped or altered flower color NGI plants of the present invention. If the NGI plant is treated with a chemical mutagen, it may be treated with alkylating sulphonates and sulphates, such as ethyl methane sulphonate, methyl methane sulphonate and diethyl sulphate, or nitroso compounds, such as nitrosoguanidine, ethylnitrosourea and methylnitrosourea. Protocols for treating seeds with mutagens such as ethyl methane sulphonate (hereinafter “EMS”) are well established in the art. A standard procedure using Arabidopsis seed includes treatment with 0.1% to 0.3% EMS for about eight to about twelve hours. Treatment methods may include immersion of shoots, buds, seeds or cell cultures in the chemical solution. Immersion under pressure or the use of penetrating agents, such as dimethyl sulphoxide, may improve the penetration of the chemical into the plant tissue. In addition, solutions may be injected into buds or administered through cut ends of shoots.

A cutting rooted in a suitable growth medium, such as oasis, or an entire NGI plant may be irradiated or treated with a chemical mutagen using the processes described herein. If a rooted cutting is used, once irradiated or treated with a chemical mutagen, the cutting is allowed to grow. Shoots from the treated plants containing flowers having petals exhibiting the striped pattern or altered flower color described herein are then selected and propagated through cuttings or other forms of asexual reproduction. Further selections may be made if desired and maintained through successive generations via asexual reproduction. If an entire NGI plant is irradiated or treated with a chemical mutagen, shoots from the treated plant containing flowers having petals exhibiting the striped pattern or altered flower color are selected and propagated through asexual reproduction.

Using the techniques described herein, the striped pattern or altered petal color may be incorporated into NGI plants having many different flower colors, including plants with bicolor flowers. Additionally, the striped pattern or altered flower color can be incorporated into NGI plants having solid green foliage, green and yellow variegated foliage, dark green foliage, dark purplish leaves, dark purplish and cream variegated foliage, etc.

The NGI plants of the present invention are genetically stable and can be stably reproduced by means of asexual propagation. It is expected that any striped or altered flower color NGI plant can be produced commercially through asexual propagation. Cuttings for asexual propagation can be taken at any time of the year and no special hormones or soil mixtures are required.

By way of example, and not of limitation, examples of the present invention shall now be given.

### EXAMPLE 1

### Irradiation Program to develop NGI Plant 1865

‘Grape Crush’ (U.S. Plant Pat. No. 10,107) is a compact, New Guinea impatiens plant having medium green foliage and vibrant solid red-purple flowers. The petals are brighter than Red-Purple Group 74A with the lower four petals commonly displaying bases of Red Group 53C (adaxial), and Red-Purple Group 74B (abaxial). As shown in FIG. 1, the petals of ‘Grape Crush’ do not exhibit a striped pattern. ‘Grape Crush’ is commercially available from Ball FloraPlant, 622 Town Road, West Chicago, Ill. 60185-2698.

Cuttings from ‘Grape Crush’ were irradiated with 2 kilorads of gamma rays. The irradiated cuttings were allowed to grow. A cutting containing a flower having petals which exhibited a striped pattern was made and allowed to grow. The resulting plant contained a number of flowers having petals which exhibited the striped pattern. A cutting of these flowers containing the petals with the striped pattern was made and planted. This cutting was designated 1865. As shown in FIG. 2, the petals of 1865 have a color of Red-Purple Group 74A (adaxial) and 74B (abaxial). The stripes have a color of Red-Purple Group 65C.

### EXAMPLE 2

### Detailed Description of Striped NGI Plant 1865 and Comparison with ‘Grape Crush’

The color chart used in the identification of colors described herein is the R.H.S. Colour Chart of The Royal Horticultural Society, London, England.

| Characteristic | NGI Plant 1865 | ‘Grape Crush’ |
|---|---|---|
| Propagation | | |
| Type cutting | Terminal tip | Terminal tip |
| Time to initiate | Approximately 14-21 days | Approximately 14-21 days |
| roots | | |
| Rooting habit | Fibrous, branching | Fibrous, branching |
| Plant Description | | |
| General appearance | Medium, upright mounded | Moderately compact, upright |
| and form | | mounded |
| Crop time | Finished plant 10 weeks after | Finished plant 8 to 10 weeks after |
| | planting a rooted cutting | planting a rooted cutting |
| Branching habit | Freely basal branching | Freely basal branching without |
| | without pinching or growth | pinching or growth regulators |
| | regulators | |
| Lateral branch | Approximately 4 | Approximately 5 |
| number | | |
| Lateral branch | Approximately 11.8 cm | Approximately 11.5 cm |
| length | | |
| Lateral branch | Approximately 8 mm | Approximately 8 mm |
| diameter | | |
| Internode length | Approximately 4.6 cm | Approximately 2.5 to 2.8 cm |
| Stem color | Greener than Yellow-Green | Greener than Yellow-Green |
| | Group 144A with Greyed | Group 144A with Greyed- |
| | Purple Group 183B at nodes | Purple Group 183D at nodes. |
| Height of foliage | Approximately 16 cm above a 10 | Approximately 8 to 9 cm |
| | cm pot | above a 10 cm pot |
| Area of spread | Approximately 27 cm | Approximately 20 to 23 |
| Foliage Description | | |
| Form | Elliptic with acuminate apex | Elliptic with acuminate apex |
| | and attenuate base | and an acuminate base |
| Margin | Serrate, ciliate | Serrate |
| Arrangement | Alternate or in whorles of 5 or 6 | Alternate or in whorles of 5 or 6 |
| Venation pattern | Arcuate | Arcuate |
| Surface | Upper is rough; lower smooth | Upper is rough; lower smooth |
| Color of mature | Between Green Group 137 A and | Green Group 139A with veins |
| foliage-upper | Green Group 143A with veins of | and midrib of Yellow-Green |
| surface | Yellow-Green Group 148A and | Group 146D |
| | midrib of Greyed-Orange Group | |
| | 76C | |
| Color of mature | Between Yellow-Green Group | Green Group 138A with midrib |
| foliage-lower | 146B and Yellow-Green Group | and veins of Yellow-Green Group |
| surface | 147B with veins and midrib of | 146D |
| | Yellow-Green Group 148A | |
| Size | Approximately 9.2 cm in length | Approximately 9 to 10 cm in |
| | and approximately 3.1 cm in width | length and 3 to 3.7 cm in width |
| Petiole length | Approximately 1.8 cm | Approximately 2 cm |
| Petiole diameter | Approximately 2 mm | Approximately 2 mm |
| Petiole color | Lighter than Grey-Orange Group | Lighter than Grey-Orange Group |
| | 176C | 176C |
| Flower Description | | |
| Flowering habit | Freely flowering under outdoor | Freely flowering under outdoor |
| | conditions with substantially | conditions with substantially |
| | continuous blooming from spring | continuous blooming from spring |
| | and fall | until fall |
| Flowers borne | Above foliage, arising from leaf | Above foliage, arising from leaf |
| | axils | axils |
| Peduncle length | Approximately 6.1 cm | Approximately 6 cm |
| Peduncle color | Yellow-Green Group 146C | Yellow-Green Group 146C with |
| | overlaid with Greyed-Purple | very faint overlay of Greyed- |
| | Group 183D | Purple Group 183D |
| Flower form | Single | Single |
| Quantity of flowers | Approximately 3 per lateral branch | Approximately 6 to 7 per lateral |
| | | branch |
| Flower size | Approximately 5.9 cm | Approximately 5.3 to 6 cm |
| Number of petals | 5, overlapping | 5, overlapping |
| Petal texture | Iridescent | Iridescent |
| Petal shape | Obovate | Somewhat heart-shaped |
| Petal margin | Entire | Entire |
| Petal apex | Emarginate | Emarginate |
| Petal base | Lower petals narrow; upper petal | Lower petals narrow; upper petal |
| | has a broad base | has a broad base |
| Petal length | 3.3 cm | 3 cm |
| Petal width | 3.5 cm | 3.7 cm |
| Flower color-fully | Petals are Red-Purple Group 74A | Petals are brighter than Red- |
| opened | with stripes of Red-Purple Group | Purple Group 74A with the lower |
| | 65C (adaxial); Red-Purple Group | four petals commonly displaying |
| | 74B (abaxial) | bases Red Group 53A (adaxial); |
| | | and Red-Purple Group 74B |
| | | (abaxial) |
| Flower bud shape | Ellipsoidal | Ellipsoidal |
| Flower bud length | Approximately 2.1 cm | Approximately 2 cm |
| Flower bud diameter | Approximately 1.1 cm | Approximately 1.7 cm |
| Sepals | Three sepals plus two rudimentary | Three sepals plus two |
| | sepals are fused into the under | rudimentary sepals are fused into |
| | surface of the superior petal. A | the under surface of the superior |
| | spur originating from the base of | petal. A spur originating from the |
| | the inferior sepal is approximately | base of the inferior sepal is |
| | 5.7 cm in length on fully opened | approximately 5 to 6 cm in length |
| | flowers. The spur color is Red- | on fully opened flowers. The spur |
| | Purple Group 60A with Yellow- | color is Red-Purple Group 60A |
| | Green Group 145A at tip. | |
| Reproductive organs | The anthers are fused together | The anthers are fused together |
| | forming one organ that surrounds | forming one organ that surrounds |
| | the pistil. The pistil is | the pistil. The stamens are Red- |
| | approximately 5 mm long, the | Purple Group 74B. The ovary |
| | stigma color is Yellow-Green | color is Yellow-Green Group |
| | Group 144A. Generally, the | 144A. Generally, the anthers shed |
| | anthers shed pollen prior to the | pollen prior to the stigma |
| | stigma becoming receptive. The | becoming receptive. The pollen |
| | pollen color is Yellow Group 11B. | color is Yellow Group 11B. |

### EXAMPLE 3

### Irradiation Program to Develop NGI Plant 2581

‘Lavender Glow’ is an upright, mounded New Guinea impatiens plant having medium green foliage and solid purple flowers. The petals have a color of Purple-Violet Group 81C (adaxial) and 81D (abaxial). As shown in FIG. 3, the petals of ‘Lavender Glow’ do not exhibit a striped pattern. ‘Lavender Glow’ is commercially available from Ball FloraPlant, 622 Town Road, West Chicago, Ill. 60185-2698.

Cuttings from ‘Lavender Glow’ were irradiated with 2 kilorads of gamma rays. The irradiated cuttings were allowed to grow. A cutting containing a flower having petals which exhibited a striped pattern was made and allowed to grow. This cutting was designated 2581. As show in FIG. 4, the petals of 2581 have a color of Purple-Violet Group 81C (adaxial) and Purple-Violet Group 81D (abaxial). The stripes have a color of Purple Group 75D.

### EXAMPLE 4

### Detailed Description of Striped NGI Plant 2581 and Comparison with ‘Lavender Glow’

| Characteristic | NGI Plant 2581 | ‘Lavender Glow’ |
|---|---|---|
| Propagation | | |
| Type cutting | Terminal tip | Terminal tip |
| Time to initiate roots | Approximately 14-21 days | Approximately 14-21 days |
| Rooting habit | Fibrous, freely branching | Fibrous, fine, freely branching |
| Plant Description | | |
| General appearance and | Upright and mounded; medium | Upright and mounded; medium |
| form | size; vigorous | size; vigorous |
| Crop time | Finished plant 8-10 weeks after | Finished plant approximately 10 |
| | planting a rooted cutting | weeks after planting a rooted |
| | | cutting |
| Branching habit | Freely basal branching without | Freely basal branching without |
| | pinching or growth regulators | pinching or growth regulators |
| Lateral branch numbers | Approximately 5 | Approximately 5 to 7 |
| Lateral branch length | Approximately 15.8 cm | Approximately 11 to 12 cm |
| Lateral branch diameter | Approximately 8.3 mm | Approximately 5 to 7 mm |
| Internode length | Approximately 4.5 cm | Approximately 3 to 4 cm |
| Stem color | Greyed-Purple Group 183A | Greyed-Purple Group 183D |
| Height of foliage | Approximately 18 cm above a | Approximately 14 to 16 cm |
| | 10 cm pot | above a 10 cm pot |
| Area of spread | Approximately 35 cm | Approximately 20 to 25 cm |
| Foliage Description | | |
| Form | Elliptic with acute apex and | Elliptic with acuminate apex |
| | acuminate base | and attenuate base |
| Margin | Serrate, ciliate | Serrate, ciliate |
| Arrangement | Opposite | Whorles or opposite |
| Venation pattern | Arcuate | Arcuate |
| Surface | Smooth | Slightly rugose; glossy; |
| | | glabrous |
| Color of mature foliage- | Closest to Green Group 137C | Green Group 139A with veins |
| upper surface | with veins of Greyed-Purple | of Red-Purple Group 59C |
| | Group 185B | |
| Color of mature foliage- | Closest to Green Group 137C | Green Group 138B with veins |
| lower surface | with veins of Greyed-Purple | of Red-Purple Group 59B |
| | Group 185A | |
| Size | Approximately 9.4 cm in | Approximately 7 to 8 cm in |
| | length and approximately 3.5 | length and approximately 2 to 3 |
| | cm in width | cm in width |
| Petiole length | Approximately 1.7 cm | Approximately 5 to 10 mm |
| Petiole diameter | Approximately 3 mm | Approximately 1.5 to 2.5 mm |
| Petiole color | Upper surface is Greyed- | Greyed-Purple Group 183D |
| | Purple Group 185B; lower | |
| | surface is Greyed-Purple | |
| | Group 185A | |
| Flower Description | | |
| Flowering habit | Freely flowering under outdoor | Freely flowering under outdoor |
| | conditions with substantially | conditions with substantially |
| | continuous blooming from | continuous blooming from |
| | spring until fall | spring until fall |
| Flower type | Generally flat and rounded; | Generally flat and rounded; |
| | typically face outward to | typically face outward to |
| | upward | upward |
| Flowers borne | Above foliage, arising from | Above foliage, arising from leaf |
| | leaf axils | axils |
| Peduncle length | Approximately 5.5 cm | Approximately 4 to 6 cm |
| Peduncle color | Grey-Purple Group 182B | Greyed-Purple Group 183D |
| Flower form | Single; sometimes exhibits | Single |
| | “siamese” flowers | |
| Quantity of flowers per | Approximately 3 | Approximately 2 |
| lateral stem | | |
| Quantity of flowers buds | Approximately 5 | Approximately 5 |
| per lateral stem | | |
| Quantity of flowers per | Approximately 15 | Approximately 10 |
| plant | | |
| Flower size | Approximately 6.2 cm width; | Approximately 7.3 to 7.5 cm |
| | approximately 2 cm depth | width; approximately 1 cm |
| | | depth |
| Number of petals | 5, overlapping | 5, overlapping |
| Petal texture | Smooth, iridescent | Smooth, iridescent |
| Petal shape | Obovate | Cordate |
| Petal margin | Somewhat lacerate | Entire |
| Petal apex | Emarginate | Retuse |
| Petal base | Acuminate | Attenuate |
| Petal length | Approximately 3.4 cm | Approximately 3 to 4 cm |
| Petal width | Approximately 4.2 cm | Approximately 5 to 6 cm |
| Flower color- fully opened | Petals are Purple-Violet Group | Petals are Purple-Violet Group |
| | 81C with stripes of Purple | 81C fading to Purple Group |
| | Group 75D (adaxial); Purple- | 76A (adaxial); Purple-Violet |
| | Violet Group 81D (abaxial) | Group 81D (abaxial) |
| Flower bud shape | Oval-pointed | Elliptic |
| Flower bud length | Approximately 2.4 cm | Approximately 1.6 to 1.8 cm |
| Flower bud diameter | Approximately 1.3 cm | Approximately 1 cm |
| Flower bud color | Purple-Violet Group 81b | Purple-Violet Group 80A |
| Sepals | Three sepals plus two | Three sepals plus two |
| | rudimentary sepals are fused | rudimentary sepals are fused |
| | into the under surface of the | into the under surface of the |
| | superior petal. A spur | superior petal. A spur |
| | originating from the base of the | originating from the base of the |
| | inferior sepal is approximately | inferior sepal is approximately 5 |
| | 5.3 cm in length on fully | to 6 cm in length on fully |
| | opened flowers. The spur color | opened flowers. The spur color |
| | is Red-Purple Group 60A. | is Greyed-Purple 183D |
| Reproductive organs | The anthers are fused together | The anthers are fused together |
| | forming one organ that | forming one organ that |
| | surrounds the pistil. The pistil | surrounds the pistil. The ovary |
| | is approximately 5 mm long, | color is Yellow-Green Group |
| | the stigma color is Yellow- | 144A. Anthers are obovate. |
| | Green Group 150D, the style | Generally, the anthers shed |
| | color is Green Group 143C, | pollen prior to the stigma |
| | and the ovary color is Green | becoming receptive. Pollen |
| | Group 143A. Anthers are 3 | color is Yellow Group 11B. |
| | mm in length having a color of | |
| | Yellow Group 12D. Generally, | |
| | the anthers shed pollen prior to | |
| | the stigma becoming receptive. | |
| | Pollen is produced in moderate | |
| | amounts and has color Yellow | |
| | Group 11C. | |

### EXAMPLE 5

### Irradiation Program to Develop NGI Plant 2582

‘Lavender Glow’ is an upright, mounded New Guinea impatiens plant having medium green foliage and solid red-purple flowers. The petals have a color of Purple-Violet Group 81C (adaxial) and Purple-Violet Group 81D (abaxial). As shown in FIG. 3, the petals of ‘Lavender Glow’ do not exhibit a striped pattern. Lavender glow is commercially available from Ball FloraPlant, 622 Town Road, West Chicago, Ill. 60185-2698.

Cuttings from ‘Lavender Glow’ were irradiated with 2 kilorads of gamma rays. The irradiated cuttings were allowed to grow. A cutting containing a flower having petals which exhibited an altered flower color was made and allowed to grow. This cutting was designated 2582. As shown in FIG. 5, the petals of 2582 have a color of Purple Group 75D with Red-Purple Group 64C at the base (adaxial). Purple Group 75D with Red-Purple Group 64C at the base with midveins of Red-Purple Group 64B (abaxial).

### EXAMPLE 6

### Detailed Description of Altered Flower Color NGI Plant 2582 and Comparison with ‘Lavender Glow’

| Characteristic | NGI Plant 2582 | ‘Lavender Glow’ |
|---|---|---|
| Propagation | | |
| Type cutting | Terminal tip | Terminal tip |
| Time to initiate roots | Approximately 14-21 days | Approximately 14-21 days |
| Rooting habit | Fibrous, freely branching | Fibrous, fine, freely branching |
| Plant Description | | |
| General appearance and form | Upright and mounded; | Upright and mounded; medium |
| | medium size; vigorous | size; vigorous |
| Crop time | Finished plant 8-10 weeks | Finished plant approximately |
| | after planting a rooted | 10 weeks after planting a |
| | cutting | rooted cutting |
| Branching habit | Freely basal branching | Freely basal branching without |
| | without pinching or growth | pinching or growth regulators |
| | regulators | |
| Lateral branch number | Approximately 4 | Approximately 5 to 7 |
| Lateral branch length | Approximately 15.3 cm | Approximately 11 to 12 cm |
| Lateral branch diameter | Approximately 8.3 mm | Approximately 5 to 7 mm |
| Internode length | Approximately 5 cm | Approximately 3 to 4 cm |
| Stem color | Greyed-Purple Group 183A | Greyed-Purple Group 183D |
| Height of foliage | Approximately 21 cm above | Approximately 14 to 16 cm |
| | a 10 cm pot | above a 10 cm pot |
| Area of spread | Approximately 31 cm | Approximately 20 to 25 cm |
| Foliage Description | | |
| Form | Elliptic with acute apex and | Elliptic with acuminate apex |
| | acuminate base | and attenuate base |
| Margin | Serrate, ciliate | Serrate, ciliate |
| Arrangement | Opposite | Whorles or opposite |
| Venation pattern | Arcuate | Arcuate |
| Surface | Smooth | Slightly rugose; glossy; |
| | | glabrous |
| Color of mature foliage- | Closest to Green Group | Green Group 139A with veins |
| upper surface | 139A with veins of Greyed- | of Red-Purple Group 59C |
| | Purple Group 185C | |
| Color of mature foliage- | Closest to Green Group | Green Group 138B with veins |
| lower surface | 137C with veins of Greyed- | of Red-Purple Group 59B |
| | Purple Group 185A | |
| Size | Approximately 7.5 cm in | Approximately 7 to 8 cm in |
| | length and approximately 3.3 | length and approximately 2 to |
| | cm in width | 3 cm in width |
| Petiole length | Approximately 1.4 cm | Approximately 5 to 10 mm |
| Petiole diameter | Approximately 2 mm | Approximately 1.5 to 2.5 mm |
| Petiole color | Upper surface is Greyed- | Greyed-Purple Group 183D |
| | Purple Group 185B; lower | |
| | surface is Greyed-Purple | |
| | Group 185A | |
| Flower Description | | |
| Flowering habit | Freely flowering under | Freely flowering under outdoor |
| | outdoor conditions with | conditions with substantially |
| | substantially continuous | continuous blooming from |
| | blooming from spring until | spring until fall |
| | fall | |
| Flower type | Generally flat and rounded; | Generally flat and rounded; |
| | typically face outward to | typically face outward to |
| | upward | upward |
| Flower borne | Above foliage, arising form | Above foliage, arising from |
| | leaf axils | leaf axils |
| Peduncle length | Approximately 5.1 cm | Approximately 4 to 6 cm |
| Peduncle color | Greyed-Red Group 182B | Greyed-Purple Group 183D |
| Flower form | Single | Single |
| Quantity of flowers per | Approximately 2 | Approximately 2 |
| lateral stem | | |
| Quantity of flowers buds per | Approximately 4 | Approximately 5 |
| lateral stem | | |
| Quantity of flowers per plant | Approximately 8 | Approximately 10 |
| Flower size | Approximately 6.5 cm width; | Approximately 7.3 to 7.5 |
| | approximately 2 cm depth | width; approximately 1 cm |
| | | depth |
| Number of petals | 5, overlapping | 5, overlapping |
| Petal texture | Smooth, iridescent | Smooth, iridescent |
| Petal shape | Obovate | Cordate |
| Petal margin | Entire | Entire |
| Petal apex | Emarginate | Retuse |
| Petal base | Acuminate | Attenuate |
| Petal length | Approximately 3.1 cm | Approximately 3 to 4 cm |
| Petal width | Approximately 4.1 cm | Approximately 5 to 6 cm |
| Flower color-fully opened | Petals are Purple Group 75D | Petals are Purple-Violet Group |
| | with Red-Purple Group 64C | 81C fading to Purple Group |
| | at the base (adaxial); Purple | 76A (adaxial); Purple-Violet |
| | Group 75D with Red-Purple | Group 81D (abaxial) |
| | Group 64C at the base with | |
| | midveins of Red-Purple | |
| | Group 64B (abaxial) | |
| Flower bud shape | Oval-pointed | Elliptic |
| Flower bud length | Approximately 1.7 cm | Approximately 1.6 to 1.8 cm |
| Flower bud diameter | Approximately 1 cm | Approximately 1 cm |
| Flower bud color | | Purple-Violet Group 80A |
| Sepals | Three sepals plus two | Three sepals plus two |
| | rudimentary sepals are fused | rudimentary sepals are fused |
| | into the under surface of the | into the under surface of the |
| | superior petal. A spur | superior petal. A spur |
| | originating from the base of | originating from the base of the |
| | the inferior sepal is | inferior sepal is approximately |
| | approximately 5 cm in length | 5 to 6 cm in length on fully |
| | on fully opened flowers. The | opened flowers. The spur |
| | spur color is Red-Purple | color is Greyed-Purple Group |
| | Group 60A. | 183D. |
| Reproductive organs | The anthers are fused | The anthers are fused together |
| | together forming one organ | forming one organ that |
| | that surrounds the pistil. The | surrounds the pistil. The ovary |
| | pistil is approximately 5 mm | color is Yellow-Green Group |
| | long, the stigma color is | 144A. Anthers are obovate. |
| | Yellow-Green Group 144D, | Generally, the anthers shed |
| | the style color is Yellow- | pollen prior to the stigma |
| | Green Group 144C, and the | becoming receptive. Pollen |
| | ovary color is Yellow-Green | color is Yellow Group 11B. |
| | Group 144B. Anthers are 3 | |
| | mm in length having a color | |
| | of Yellow Group 12D. | |
| | Generally, the anthers shed | |
| | pollen prior to the stigma | |
| | becoming receptive. Pollen | |
| | is produced in moderate | |
| | amounts and has color | |
| | Yellow Group 12D. | |

The present invention is illustrated by way of the foregoing description and examples. The foregoing description is intended as a non-limiting illustration, since many variations will become apparent to those skilled in the art in view thereof It is intended that all such variations within the as defined in the following claims.

What is claimed is:

1. A New Guinea impatiens plant comprising a flower having at least one petal which exhibits a striped pattern.

2. The New Guinea impatiens plant of claim 1, wherein the plant has a pedigree which includes the plant 1865 or 2581.

3. Pollen of the plant of claim 1.

4. An ovule of the plant of claim 1.

5. A tissue culture comprising regenerable cells of the plant of claim 1.

6. A cutting of the plant of claim 1.

7. A method for producing a New Guinea impatiens plant having a flower with at least one petal which exhibits a striped pattern, the method comprising the steps of: irradiating a New Guinea impatiens plant with electromagnetic radiation or ion beams and selecting a shoot from said plant containing a flower having at least one petal which exhibits a striped pattern.

8. The method of claim 7 wherein the New Guinea impatiens plant is a cutting or a whole plant.

9. The method of claim 7 wherein the New Guinea impatiens plant is irradiated with gamma rays, x-rays or ultraviolet rays.

10. The method of claim 7 wherein the New Guinea impatiens plant is irradiated with from about 1.5 to about 3.5 krads of electromagnetic radiation or ion beams.

11. A New Guinea impatiens plant comprising a flower having at least one petal which exhibits a striped pattern produced by the method of claim 7.

12. Pollen of the plant of claim 11.

13. An ovule of the plant of claim 11.

14. A tissue culture comprising regenerable cells of the plant of claim 11.

15. A cutting of the plant of claim 11.

16. A method for producing a New Guinea impatiens plant having a flower with at least one petal which exhibits a striped pattern, the method comprising the steps of: treating a New Guinea impatiens plant with a chemical mutagen and selecting a shoot from said plant containing a flower having at least one petal which exhibits a striped pattern.

17. The method of claim 16 wherein the New Guinea impatiens plant is a cutting or a whole plant.

18. The method of claim 16 wherein the chemical mutagen is ethyl methane sulphonate, methyl methane sulphonate, diethyl sulphate, nitrosoguanidine, ethylnitrosourea or methylnitrosourea.

19. A New Guinea impatiens plant comprising a flower having at least one petal which exhibits a striped pattern produced by the method of claim 16.

20. Pollen of the plant of claim 19.

21. An ovule of the plant of claim 19.

22. A tissue culture comprising regenerable cells of the plant of claim 19

23. A cutting of the plant of claim 19.

24. A method for altering the color of the petals of a flower of a New Guinea impatiens plant, the method comprising the steps of: treating a New Guinea impatiens plant with electromagnetic radiation or ion beams and selecting a shoot from said plant containing a flower having at least one petal exhibiting an altered color.

25. The method of claim 24 wherein the New Guinea impatiens plant is a cutting or a whole plant.

26. The method of claim 24 wherein the New Guinea impatiens plant is irradiated with gamma rays, x-rays or ultraviolet rays.

27. The method of claim 24 wherein the New Guinea impatiens plant is irradiated with from about 1.5 to about 3.5 krads of electromagnetic radiation or ion beams.

28. A New Guinea impatiens plant comprising a flower having at least one petal which exhibits an altered color produced by the method of claim 24.

29. The New Guinea impatiens plant of claim 28, wherein the plant has a pedigree which includes the plant 2582.

30. Pollen of the plant of claim 28.

31. An ovule of the plant of claim 28.

32. A tissue culture comprising regenerable cells of the plant of claim 28.

33. A cutting of the plant of claim 28.

34. A method for altering the color of the petals of a flower of a New Guinea impatiens plant, the method comprising the steps of: treating a New Guinea impatiens plant with a chemical mutagen and selecting a shoot from said plant containing a flower having at least one petal exhibiting an altered color.

35. The method of claim 34 wherein the chemical mutagen is ethyl methane sulphonate, methyl methane sulphonate, diethyl sulphate, nitrosoguanidine, ethylnitrosourea or methylnitrosourea.

36. A New Guinea impatiens plant comprising a flower having at least one petal which exhibits an altered color produced by the method of claim 34.

37. Pollen of the plant of claim 36.

38. An ovule of the plant of claim 36.

39. A tissue culture comprising regenerable cells of the plant of claim 36.

40. A cutting of the plant of claim 36.
